(19) United States
(12) Patent Application Publication
(10) Pub. No.: US 2026/0166285 A1
(43) Pub. Date: Jun. 18, 2026

(54) SHUNT FLUSHING SYSTEMS AND METHODS

(71) Applicant: ANUNCIA MEDICAL, INC., Scottsdale, AZ (US)

(72) Inventors: Marc-Alan Levine, Pottstown, PA (US); David A. Watson, San Jose, CA (US); Jacob Schuler, Tempe, AZ (US); Elsa Chi Abruzzo, Paradise Valley, AZ (US); David Szabo, Phoenix, AZ (US)

(73) Assignee: ANUNCIA MEDICAL, INC., Scottsdale, AZ (US)

(21) Appl. No.: 19/134,328

(22) PCT Filed: Nov. 29, 2023
(86) PCT No.: PCT/IB2023/062022

§ 371 Date: May 30, 2025

Related U.S. Application Data
(60) Provisional application No. 63/385,712, filed on Dec. 1, 2022.

(51) Int. Cl. A61M 27/00; A61M 39/00; A61M 39/02

(52) U.S. Cl. CPC A61M 27/006; A61M 39/0247; A61M 2039/0009; A61M 2039/0205; A61M 2039/0276; A61M 2039/0282; A61M 2205/0216; A61M 2205/04; A61M 2205/3344; A61M 2210/04; A61M 2210/0687

(57) ABSTRACT

A shunt system disposed beneath a patient's skin to shunt fluid from tissue site located upstream to a drain site, comprising a flush assembly having a depressible dome comprised of a flexible material and a base forming a flush cavity, the depressible dome and the base creating a collapsible fluid pathway. The depressible dome and the base may comprise a proximal connector at a proximal end connected to a fluid collection tube, and a drain tube connected at a distal end, wherein the fluid collection tube, the flush assembly, and the drain tube create a fluid path from the tissue site to the drain site. When the depressible dome is subjected to pressure, it first obstructs the collapsible fluid pathway and with continued pressure generates a back pressure forcing fluid within the flush cavity back to the tissue site through the fluid collection tube, clearing debris.

### CROSS REFERENCE TO RELATED APPLICATIONS

This Patent Application is an application claiming priority under 35 U.S.C. §§ 111(a) and 365(a) to PCT/IB2023/062022, entitled “SHUNT FLUSHING SYSTEMS AND METHODS,” filed Nov. 29, 2023, which is related to and claims priority under 35 U.S.C. § 119(e) to U.S. Provisional Patent Application No. 63/385,712, entitled “SHUNT FLUSHING SYSTEMS AND METHODS,” filed Dec. 1, 2022, both which are incorporated by reference in its entirety for all purposes.

### FIELD

Systems and methods for shunting fluid.

### BACKGROUND

Shunts are used to transport body fluids from one region of the body to another region of the body. Shunts used in the treatment of hydrocephalus drain excess cerebrospinal fluid from a patient tissue site, such as the ventricles of the brain, to a target site, such as the abdomen, to relieve fluid pressure on the brain. Common shunts are implanted beneath the skin and have a valve member that acts as a one-way valve whereby the fluid is allowed to drain only in the direction from the tissue site to the drain site. However, after a period of use, solids and components within the fluid, such as cellular and proteinaceous components, can adhere to the tubes and accumulate, thereby restricting flow of fluid into and through the valve member, necessitating removal can accumulate and restrict flow of fluid into and through the valve member, necessitating removal and replacement of the shunt, exposing the patient to unnecessary risks associated with such surgery, and increasing the cost of treatments using shunts.

### SUMMARY

In general, the present disclosure provides a shunt system disposed beneath a patient's skin to shunt fluid from tissue site located upstream to a drain site located downstream, comprising a flush assembly having a depressible dome comprised of a flexible material and a base to form a flush cavity, the depressible dome and the base creating a collapsible fluid pathway therebetween. The depressible dome and the base may further comprise a proximal connector at a proximal end connected to a fluid collection tube, and a drain tube connected at a distal end, wherein the fluid collection tube, the flush assembly, and the drain tube create a fluid path from the tissue site to the drain site

In accordance with various aspects, the proximal connector is connected to a fluid collection tube implanted at the tissue site, and the drain tube is implanted at the drain site, and when the depressible dome is subjected to an external pressure, it first obstructs the collapsible fluid pathway and with continued pressure generates a back pressure forcing fluid within the flush cavity back to the tissue site through the fluid collection tube, clearing debris from the fluid collection tube.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure, wherein:

FIG. 1 is a top isometric view of a flush assembly according to the disclosed subject matter.

FIG. 2 is a bottom isometric view of a flush assembly according to the disclosed subject matter.

FIG. 3A is a cross-section of the flush assembly of FIGS. 1-2 in an undepressed or “passive” state.

FIG. 3B is a cross-section of the flush assembly of FIGS. 1-2 in a depressed or “active” state.

FIG. 4 is a bottom view of the flush assembly with the base removed.

FIG. 5 is a bottom view of the upper body.

FIG. 6 is an isometric view of FIG. 2.

FIG. 7 is an isometric view of the valve and channel member.

FIG. 8 is an isometric view of the channel member.

FIG. 9 is a bottom isometric view of the valve.

FIG. 10 is an elevation view of a flush assembly according to the disclosed subject matter.

FIG. 11 is a cross-section of an embodiment of the flush assembly according to the disclosed subject matter.

FIG. 12 is a cross-section of an embodiment of the flush assembly according to the disclosed subject matter.

FIG. 13 is an enlarged view of FIG. 12.

FIG. 14 is a bottom view of an embodiment of the flush assembly with the base removed

### DETAILED DESCRIPTION

Persons skilled in the art will readily appreciate that various aspects of the present disclosure can be realized by any number of methods and systems configured to perform the intended functions. Stated differently, other methods and systems can be incorporated herein to perform the intended functions. It should also be noted that the accompanying drawing figures referred to herein are not all drawn to scale but may be exaggerated to illustrate various aspects of the present disclosure, and in that regard, the drawing figures should not be construed as limiting. Finally, although the present disclosure can be described in connection with various principles and beliefs, the present disclosure should not be bound by theory

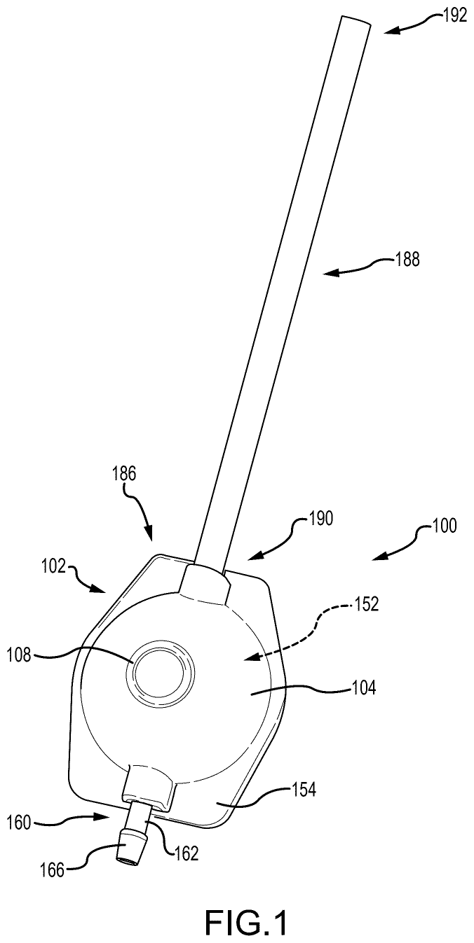

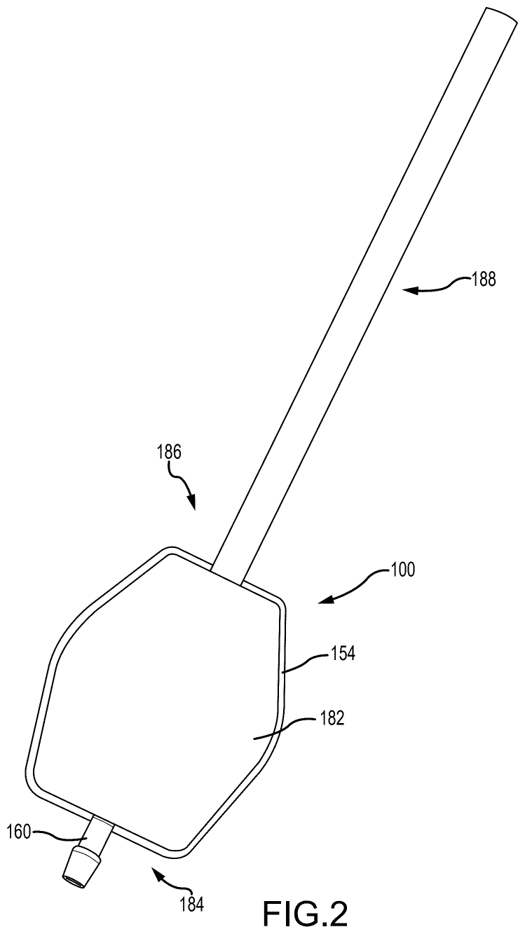

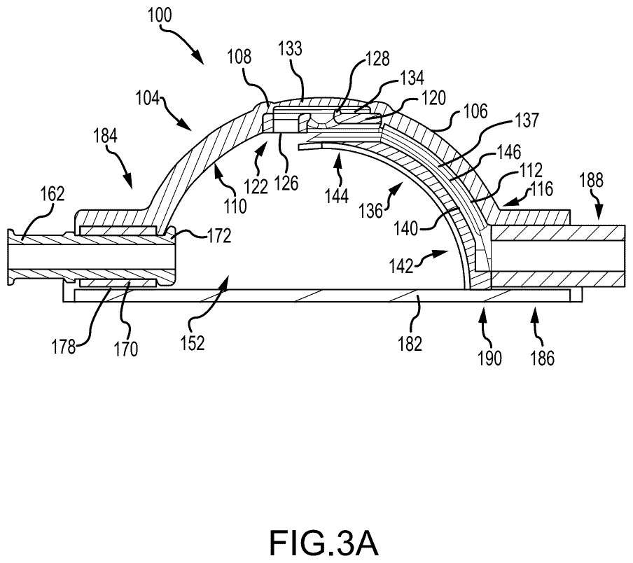

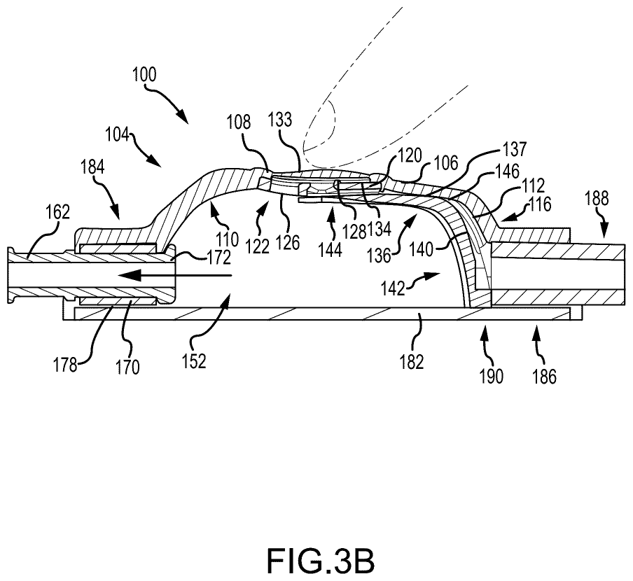

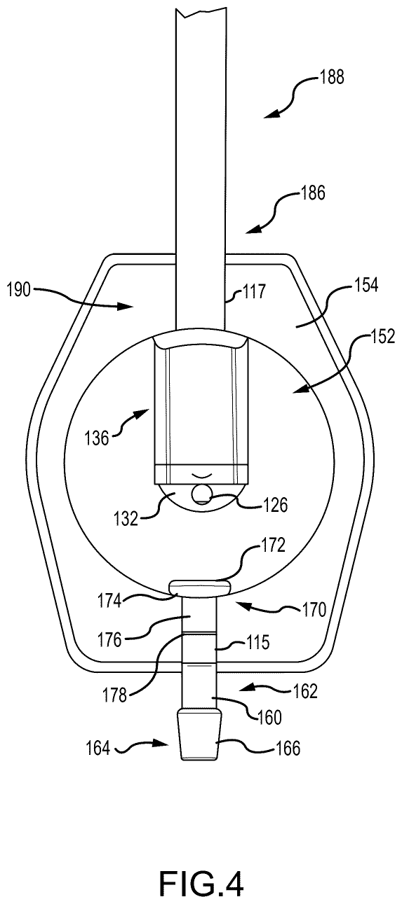

This being noted, now referring to FIGS. 1-4, an embodiment of a flush assembly 100 according to the disclosed subject matter is shown and described. The flush assembly 100 is comprised of an upper body 102, such as depressible dome 104, and a base 182, with a connector 160 at a proximal end 184, and a drain tube 188 at a distal end 186. In an implementation the flush assembly 100 is disposed beneath the skin of a patient. During use, the flush assembly 100 is used as a component of shunt system to shunt fluid from an upstream location, such as a tissue site to a downstream location, such as a drain site, and to perform retrograde prophylactic flushing (e.g., indication-based) and/or by prescribed flushing (e.g., predetermined at practitioner discretion) by allowing fluid to be forced from the flush assembly 100 to the upstream location to remove blockages in the shunt due to the accumulation of debris found in the fluid or from tissue infiltration into the drainage catheter. In an implementation, the flush assembly 100 shunts cerebrospinal fluid from the ventricles of the brain to a drainage site, typically the abdomen, in the treatment of hydrocephalus. The proximal connector 160 can be connected to a fluid collection tube or catheter implanted at the tissue site, and the drain tube 188 can be connected to other distal components of a shunt system that terminate the shunt system at a drain site. With reference specifically to FIG. 3A and as described in more detail below, when the flush assembly 100 is in a “passive” state (or undepressed) it enables fluid to flow unobstructed from the connector 160 to the drain tube 188 through a collapsible fluid pathway 137 between the depressible dome 104 and the base 182. With reference now to FIG. 3B, when the flush assembly 100 is in an “active” state (or depressed) or otherwise activated, for example, by applying an external pressure to the depressible dome (e.g., with one or more fingers), the collapsible fluid pathway 137 is obstructed and continued pressure generates a back pressure forcing fluid within a flush cavity 152 back to the tissue site proximally through the connector 160 to clear and through the fluid collection tube, clearing debris or tissue from the fluid collection tube (e.g., drainage catheter). The depressible dome 104 may be manufactured from a flexible material, such as silicone, and forms a flush cavity 152 above the base 182. By using a flexible material, the depressible dome returns to an undepressed shape after removing the external pressure. The unique integration of this collapsible fluid pathway 137 between the depressible dome 104 and the base 182 eliminates the potential for skin erosion that may occur with products of the prior art.

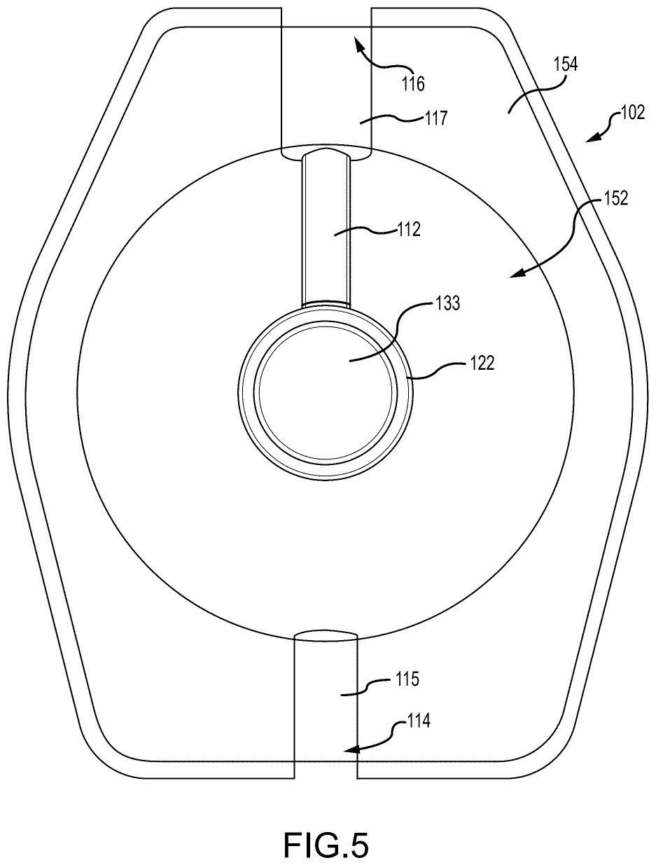

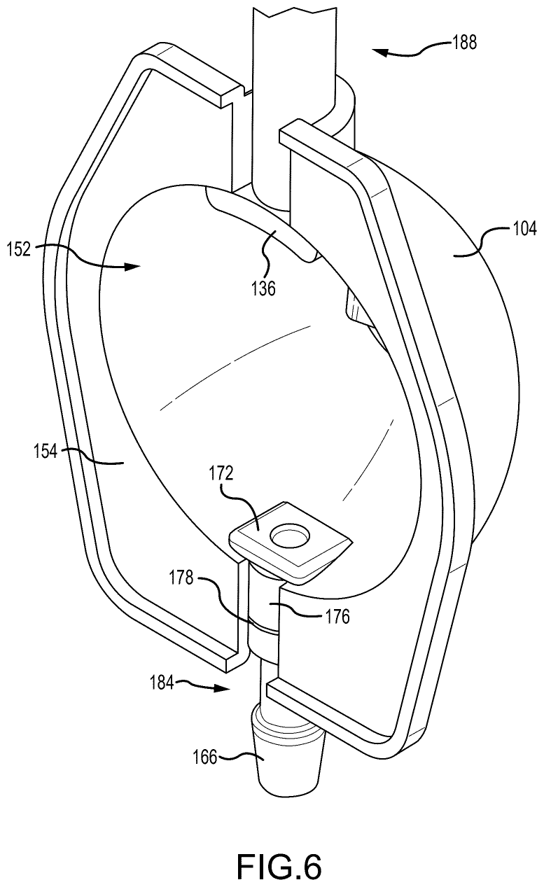

In an implementation the collapsible fluid pathway is hemispherical, alternatively the collapsible fluid pathway can have other conformations, such as generally cylindrical, hemispherical, ellipsoidal, rectangular, and/or curvilinear. The base 182 is manufactured from a resilient material, such as polyetheretherketone (PEEK), polyethylene (PE), or Acetal. Referring to FIGS. 5-6, a flanged connector 154 extends from a base portion of the dome 104 forming a proximal channel 115 extending from the flush cavity 152 to a proximal opening 114, and a distal channel 117 extending from the flush cavity 152 to a distal opening 116. The connector 160 is located at the proximal channel 115, and the drain tube 188 is located at the distal channel 117. In an embodiment, the connector 160 is formed as one piece and is integral to base 182. In another implementation, a connector 160 is not used, and a tube at the proximal channel 115 provides fluid communication between the tissue site and the flush cavity 152.

The connector 160 forms a tube 162 extending from a proximal end 164 forming a barb 166, to a head 172 at a distal end 170. The barb 166 allows the flush assembly 100 to be connected to a tube or catheter draining a tissue site. A sleeve 176 around the tube 162 positions the connector 160 within the proximal channel 115, and an O-ring 178 circumscribing the sleeve 176 creates a seal between the sleeve 176 and upper body 102. The head 172 is disposed within the flush cavity 152 preventing the connector 160 from being pulled through the proximal end 184 of the flush assembly 100 when being separated from the structure immediately upstream from the assembly 100, and when the dome 104 is depressed during a flushing operation. The head has a convex proximal face 174 conforming to the dome 104 and providing a seal there between. Alternatively, the proximal connector 160 can be sealed with a compression fit and a chemical bond and may be further reinforced with a tied filament, such as with a suture.

The drain tube 188 extends from a proximal end 190 disposed within the distal channel 117 to a distal end 192. The proximal end 190 communicates with a passage 146 formed by the dome 104 and a channel member 136. The passage 146 provides a fluid communication between the drain tube 188 and the flush cavity 152, and allows fluid draining into the flush cavity 152 from a tissue site to exit the flush assembly 100 and travel to a drain site. The passage 146 can be sealed off from the drain tube 188 by depressing the dome 104 against an occluder valve 120 disposed between the dome 104 and channel member 136. The occluder valve 120 is manufactured from a resilient material, such as silicone.

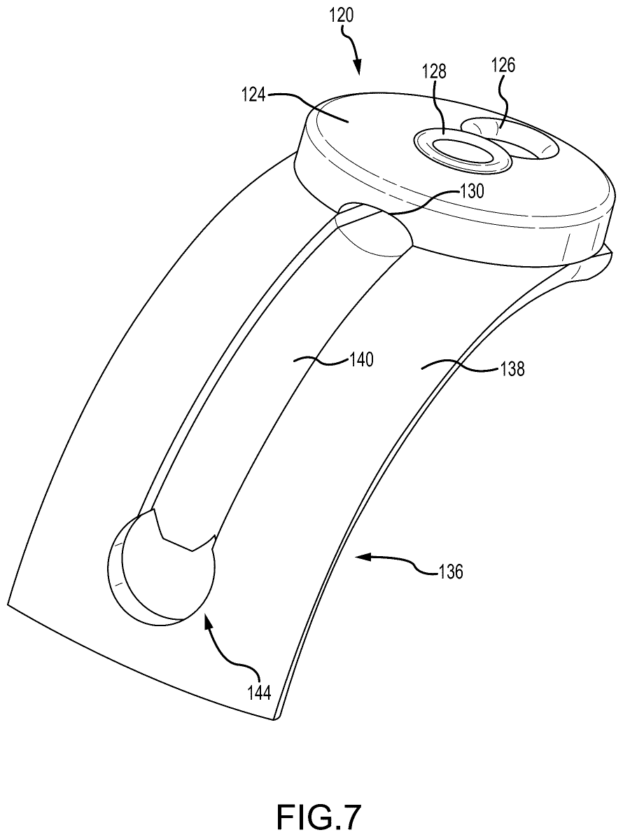

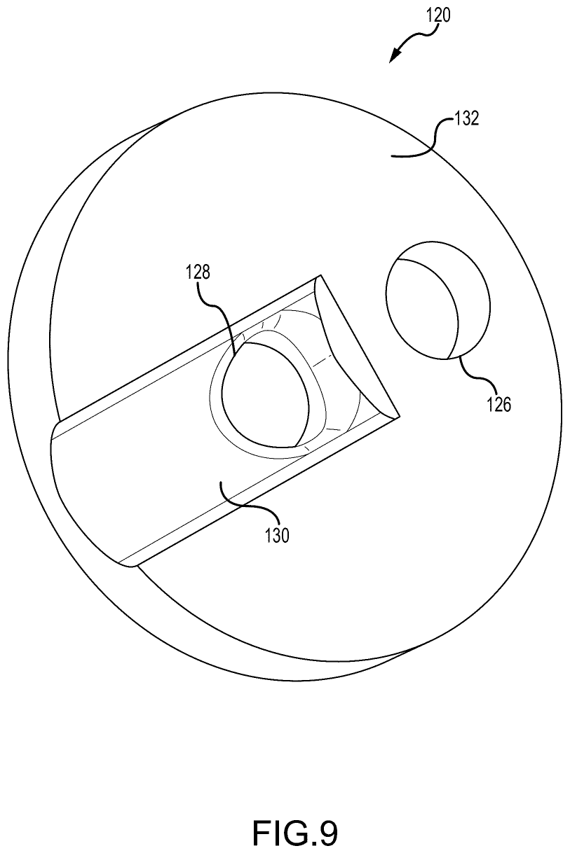

A first half of the passage 146 is formed by an upper channel 112 at an interior surface 110 of the dome 104 extending from the distal opening 116 to a valve seat 122 formed in the interior surface 110 of the central part of the dome 104. Referring to FIGS. 7 and 9, a second half of the passage 146 is formed by a lower channel 140 formed in a concave upper face 138 of the channel member 136 extending from a distal end 144 at the distal opening 116 to a proximal end 142 at the valve 120. The channel member 136 is manufactured from a flexible material, such as silicone, and the distal end 144 creates a sealing relationship with the drain tube 188 proximal end 190, and the concave upper face 138 conforms to the concave interior surface 110 of the dome 104 sealing the passage 146 from the flush cavity 152. In an implementation, the channel member 136 is a flexible tube.

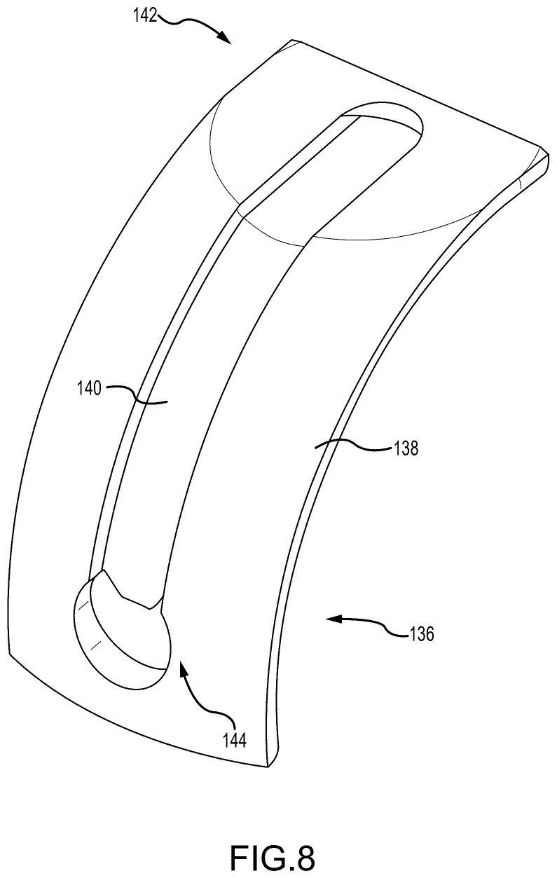

Referring to FIGS. 7 and 8, the valve 120 is a circular disc forming a centrically located downstream port 128 extending between a top surface 124 and bottom surface 132 of the disc. A valve channel 130 formed in the bottom surface 132 extends from the downstream port 128 to the edge of the valve 120. An upstream port 126 adjacent the downstream port 128 extends between the top surface 124 and bottom surface 132. The valve seat 122 receives the valve 120 whereby the bottom surface 132 around the downstream port 128 and valve channel 130 sealingly engage the proximal end 142 of the channel member 136, leaving the upstream port freely communicating with an upper chamber 134 and flush cavity 152. The outer edge of the top surface 124 of the valve 120 sealingly engages an upper rim of the valve seat 122 creating a sealing engagement therebetween. The interior surface 110 of the dome 104 is set off from the top surface 124 of the valve 120 creating the upper chamber 134.

The flush assembly 100 is implanted beneath the skin of a patient using conventional procedures known to persons of ordinary skill in the art

When not activated, the upper chamber 134 allows fluid within the flush cavity 152 to flow unrestricted through the upstream port 126 into the downstream port 128, through the passage 146, exiting the dome through the drain tube 188. When activated by either a single finger or a multi-finger pressure activation or other mechanism for depressing the depressible dome such as an actuator (e.g., servo or similar mechanism), the valve 120 closes and prevents fluid to the drain site when a pressure threshold is reached. After the pressure threshold is reached, continued pressure will expel the fluid in the flusher dome to the drain site facilitating a flushing operation. Upon the release of downward pressure from the dome 104, the elastic nature of the dome 104 allows it to rebound to its original hemispherical configuration to begin receiving fluid within the flush cavity 152 from the tissue site. In an implementation, a rate limiting mechanism within the inlet channel prevents the dome 104 from rebounding rapidly by limiting the fluid flow until a sufficient volume of fluid has filled the dome 104, or a sufficient fluid pressure within the dome 104 is achieved.

In an implementation, the dome 104, for example, on and upper surface of the dome 104, is configured to have a raised feature which can be felt on the outer surface of the patient's skin by human touch even though it is beneath the skin of the patient, allowing one to properly locate and operate the device. For example, the raised feature may be a centrally located palpation ring 108 on the exterior surface 106 of the dome 104. The palpation ring 108 is detectable (i.e., can be felt) by the user beneath the skin of the patient to properly locate and operate the device.

Either the patient, or a physician can use the flush assembly 100 to prophylactically flush the shunt to maintain the flush assembly 100 and upstream structures free from blockages, and remove debris that attach to the surfaces of the flush assembly 100 and other upstream shunt components including the drainage catheter. The prophylactic flushing can occur at any time, such as prior to, or upon, full or partial occlusion of the drainage catheter.

The flush assembly 100 can be used for access to the tissue site from outside the patient. In an implementation, the dome 104 is manufactured from a resealable material, allowing the flush assembly 100 to be used as an agent delivery pathway to the tissue site. In such an implementation, an agent can be injected into the flush cavity 152 by a needle (e.g., punctured) while still in situ beneath the skin, and the dome 104 can be depressed in the manner described above causing the agent to flow from the flush cavity 152 to the tissue site. The agents include pharmaceuticals, hydrogels, drug eluting polymers, pre-loaded bolus, antibiotics, biologics, and gene therapies and the like. In an implementation, the flush cavity 152 is accessed by a needle inserted through the dome 104.

The flush assembly 100 can be used with other shunt components, such as catheters, valves, reservoirs, and anti-siphoning devices to drain fluid from the tissue site.

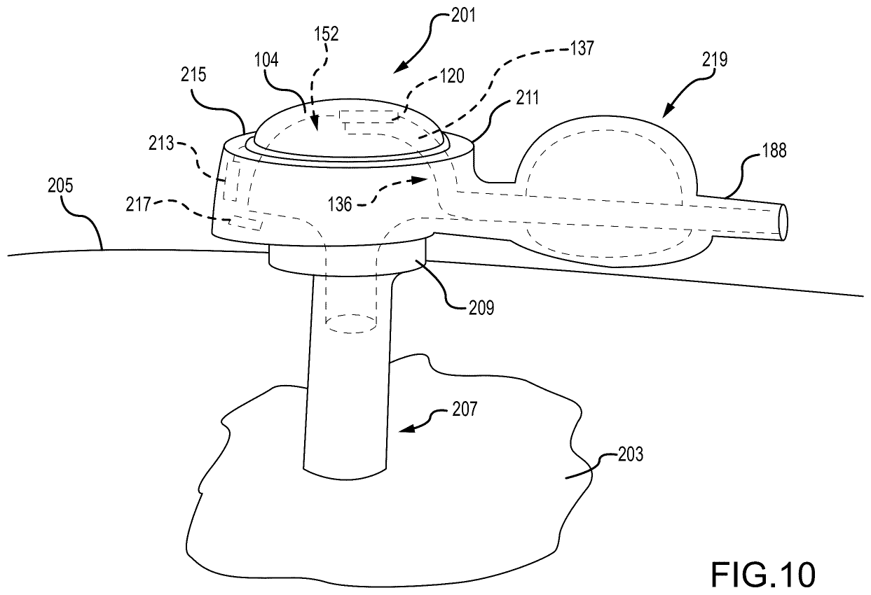

In an implementation, the flush assembly includes additional components, and the fluid pathway from the tissue site to the drain site is an angular pathway. Referring to FIG. 10, flush assembly 201 is shown connected to a tissue site, such as a ventricle 203 of the brain. The tissue site is accessed via a burr hole in the skull 205 of a patient. The flush assembly 201 forms an approximately 90-degree angle fluid pathway from the tissue site to the exterior of the skull 205. The flush assembly 201 includes features found in flush assembly 100, including a collapsible fluid pathway, such as a dome 104, a valve 120, channel member 136, and drain tube 188. Here, a catheter 207 descends from the flush assembly 201 connecting the tissue site to the flush cavity 152. In an implementation, the catheter 207 includes a burr-hole cover 209 providing an attachment point for the base of the flush assembly 201 to the catheter 207

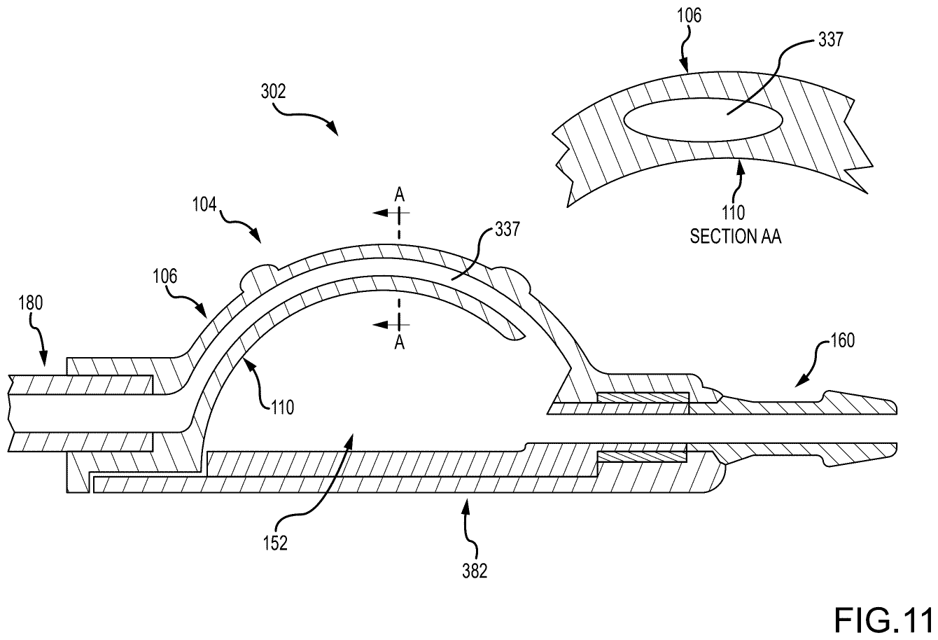

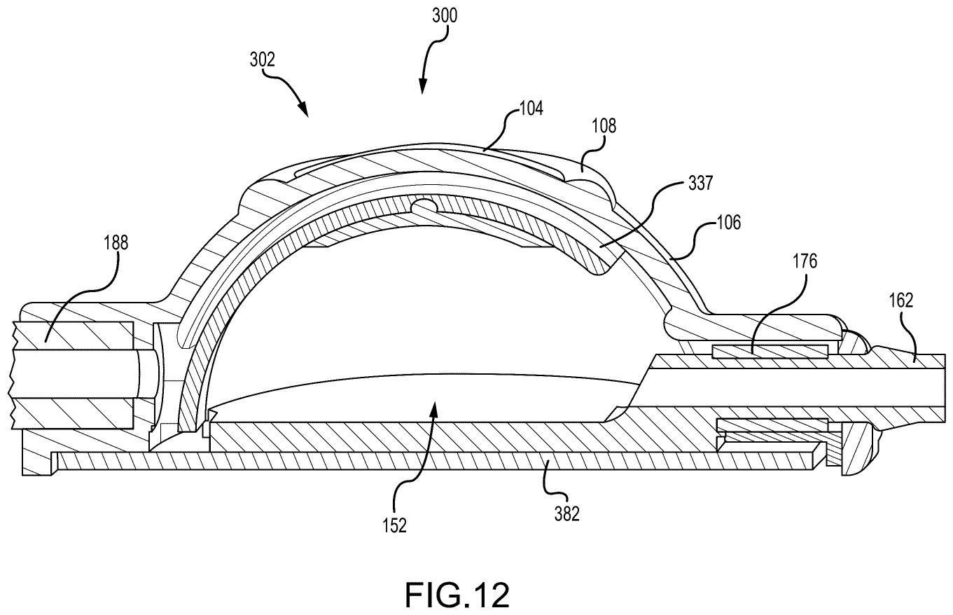

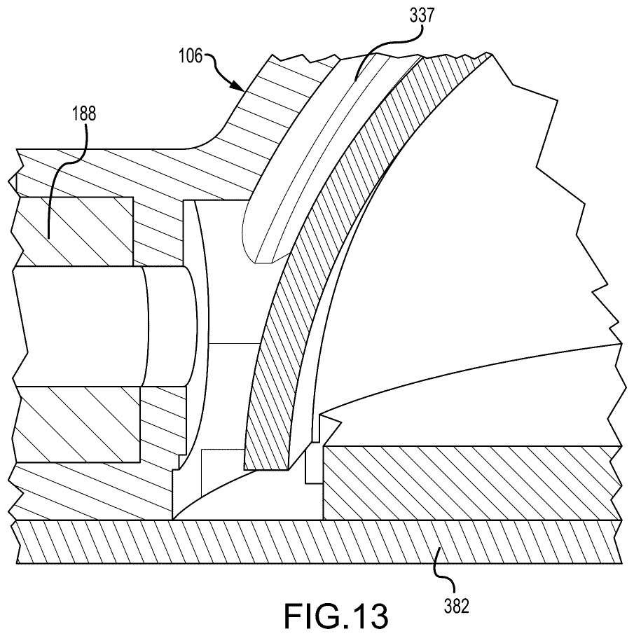

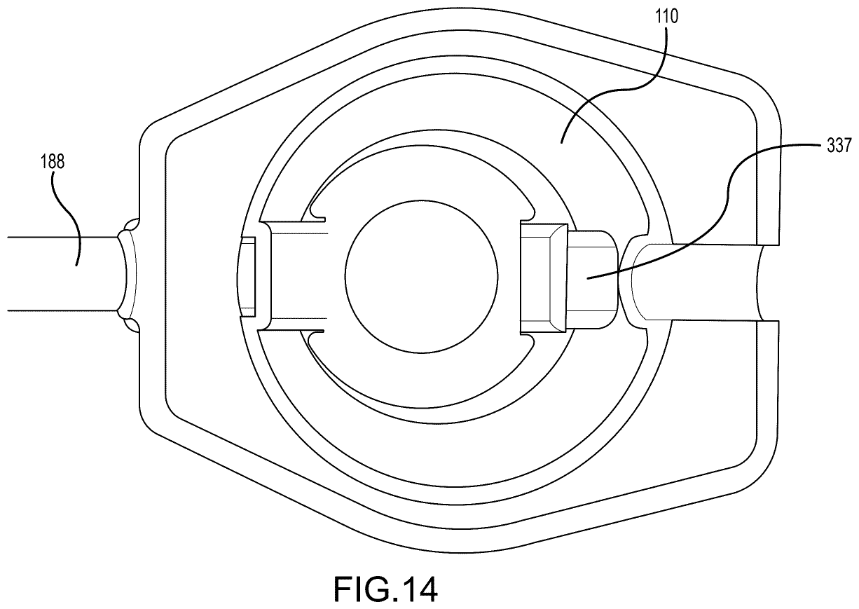

Referring to FIGS. 11-14, and embodiment of the disclosed subject matter is shown where a flush assembly 300 includes an upper body 302 and a base 382, with a collapsible fluid pathway 337 integrally molded into the dome 104 between the exterior surface 106 and interior surface 110. Fluid flows into the flush cavity 152 from the connector 160, and fluid exits the flush cavity 152 through the drain tube 188 via the collapsible fluid pathway 337. Pushing on the dome 104 from the exterior surface 106 causes the collapsible fluid pathway 337 to close, and further pressing the upper body 302 toward the base 382 causes the fluid volume within the flush cavity 152 to exit the flush assembly 300 via the connector 160, clearing debris from the drain catheter connected thereto.

As required, detailed aspects of the present disclosed subject matter are disclosed herein. However, it is to be understood that the disclosed aspects are merely exemplary of the disclosed subject matter, which may be embodied in various forms. Therefore, specific structural and functional details disclosed herein are not to be interpreted as limiting, but merely as a basis for the claims and as a representative basis for teaching one skilled in the art to variously employ the present disclosed subject matter in virtually any appropriately detailed structure.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of composition, ingredients, structure, materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

We claim:

1. A shunt system disposed beneath a patient's skin to shunt fluid from tissue site located upstream to a drain site located downstream, comprising:
a flush assembly comprising a curvilinear depressible dome comprised of a flexible material and a base to form a flush cavity, the base having a curvilinear portion corresponding to the curvilinear depressible dome to create a curvilinear collapsible fluid pathway therebetween;
the depressible dome and the base further comprising a proximal connector at a proximal end connected to a fluid collection tube, and a drain tube connected at a distal end, and wherein the fluid collection tube, the flush assembly, and the drain tube create a fluid path from the tissue site to the drain site;
wherein the proximal connector is connected to a fluid collection tube implanted at the tissue site, and the drain tube is implanted at the drain site; and
wherein depressing the depressible dome with an external pressure first obstructs the curvilinear collapsible fluid pathway and with continued pressure generates a back pressure forcing fluid within the flush cavity back to the tissue site through the fluid collection tube, clearing debris from the fluid collection tube.

2. The shunt system of claim 1, wherein the external pressure is applied by depressing the depressible dome with one or more fingers of a user.

3. The shunt system of claim 1, wherein the external pressure is applied by an actuator.

4. The shunt system of claim 1, wherein the depressible dome is manufactured from a flexible material such that the depressible dome returns to an undepressed shape after removing the external pressure.

5. The shunt system of claim 4, wherein the flexible material is silicone.

6. The shunt system of claim 1, further comprising a flanged connector extending from a base portion of the depressible dome and forming a proximal channel extending from the flush cavity to a proximal opening, and a distal channel extending from the flush cavity to a distal opening.

7. The shunt system of claim 6, wherein the proximal connector is located at the proximal channel, and the drain tube is located at the distal channel.

8. The shunt system of claim 1, wherein the proximal connector is formed as one piece and is integrated with the base.

9. The shunt system of claim 1, wherein the collapsible fluid pathway is at least one of cylindrical, hemispherical, ellipsoidal, rectangular, and curvilinear.

10. The shunt system of claim 1, wherein the base is manufactured from a resilient material.

11. The shunt system of claim 10, wherein the resilient material is one of polyetheretherketone (PEEK), polyethylene (PE), or Acetal.

12. The shunt system of claim 1, the proximal connector is sealed with a compression fit and a chemical bond and reinforced with a tied filament.

13. The shunt system of claim 1, wherein an upper surface of the flush assembly further comprises a raised feature detectable beneath patient's skin by a human touch on an outer surface of the patient's skin.

14. The shunt system of claim 1, wherein the flush cavity is comprised of a resealable material that can be punctured to allow for delivery of a therapeutic agent such as antibiotics, biologics, gene therapies, hydrogels, pharmaceuticals, hydrogels, drug eluting polymers, and pre-loaded bolus.

15. The shunt system of claim 1, wherein the flush assembly is implanted under the patient's skin directly over a burr hole in a skull of the patient forming am approximately 90-degree angle with the fluid collection tube.

16. A shunt system disposed beneath a patient's skin to shunt fluid from tissue site located upstream to a drain site located downstream, comprising:
a flush assembly comprising a depressible dome comprised of a flexible material and a base to form a flush cavity, the depressible dome and the base creating a collapsible fluid pathway therebetween;
the depressible dome and the base further comprising a proximal connector at a proximal end connected to a fluid collection tube, and a drain tube connected at a distal end, and wherein the fluid collection tube, the flush assembly, and the drain tube create a fluid path from the tissue site to the drain site;
wherein the proximal connector is connected to a fluid collection tube implanted at the tissue site and is sealed with a compression fit and a chemical bond and reinforced with a tied filament, and the drain tube is implanted at the drain site; and
wherein depressing the depressible dome with an external pressure first obstructs the collapsible fluid pathway and with continued pressure generates a back pressure forcing fluid within the flush cavity back to the tissue site through the fluid collection tube, clearing debris from the fluid collection tube.

17. The shunt system of claim 16, wherein the depressible dome is curvilinear and comprised of a flexible material and the base comprises a curvilinear portion corresponding to the curvilinear depressible dome such that the collapsible fluid pathway is curvilinear.
